(19) United States
(12) Patent Application Publication
(10) Pub. No.: US 2017/0372238 A1
(43) Pub. Date: Dec. 28, 2017

(54) Accountable Care Organization Provider Network Design - a Systematic Data-Driven Approach

(71) Applicant: International Business Machines Corporation, Armonk, NY (US)

(72) Inventors: Kathryn L. Howard, Boston, MA (US); Kun Lin, Bethesda, MD (US); Gigi Y. Yuen-Reed, Tampa, FL (US); Yuchen Zheng, Smyrna, GA (US)

(21) Appl. No.: 15/193,733

(22) Filed: Jun. 27, 2016

(51) Int. Cl. G06Q 10/06; G06F 17/30; G06F 19/00; G06Q 30/06; G06Q 50/22

(52) U.S. Cl. CPC G06Q 10/067; G06F 19/328; G06Q 30/0631; G06F 17/3053; G06F 19/327; G06Q 50/22

(57) ABSTRACT

A systematic data-driven approach for building an Accountable Care Organization (ACO) is provided. In one aspect, a method for forming an ACO includes: determining groups of healthcare providers that have x number of patients in common; detecting communities in the groups using a recursive community detection process; ranking contractual organizations of the healthcare providers based on how well the contractual organizations represent the communities; and making recommendations for the contractual organizations to include in the ACO based on the ranking. A system for forming an ACO is also provided

### FIELD OF THE INVENTION

The present invention relates to Accountable Care Organizations (ACOs), and more particularly, to a systematic data-driven approach for building an ACO.

### BACKGROUND OF THE INVENTION

In recent years, there has been an increasing emphasis on enabling value-driven healthcare, aimed at improving outcomes, lowering costs, and increasing overall access to care for patients. A prominent example of value-driven delivery systems is the formation of patient-centric Accountable Care Organizations (ACOs).

ACOs are groups of physicians, facilities, and other healthcare providers who come together to provide coordinated care to their patients. Providers belonging to these naturally-occurring preexisting networks may be more ready to be accountable for managing the health of a population by sharing the risks and benefits of being part of a shared savings program.

Thus, effective techniques for the identification of naturally-occurring ACOs are needed in transitioning a healthcare care system from fee-for-service to valued-based reimbursement.

### SUMMARY OF THE INVENTION

The present invention provides a systematic data-driven approach for building an Accountable Care Organization (ACO). In one aspect of the invention, a method for forming an ACO is provided. The method includes: determining groups of healthcare providers that have x number of patients in common; detecting communities in the groups using a recursive community detection process; ranking contractual organizations of the healthcare providers based on how well the contractual organizations represent the communities; and making recommendations for the contractual organizations to include in the ACO based on the ranking.

In another aspect of the invention, a system for forming an ACO is provided. The system includes a recommender engine that is configured to: determine groups of healthcare providers that have x number of patients in common; detect communities in the groups using a recursive community detection process; rank contractual organizations of the healthcare providers based on how well the contractual organizations represent the communities; and make recommendations for the contractual organizations to include in the ACO based on the ranking.

A more complete understanding of the present invention, as well as further features and advantages of the present invention, will be obtained by reference to the following detailed description and drawings.

### BRIEF DESCRIPTION OF THE DRAWINGS

FIG. 1 is a diagram illustrating an exemplary methodology for forming an Accountable Care Organization (ACO) according to an embodiment of the present invention;

FIG. 2 is a diagram schematically depicting the steps of FIG. 1 according to an embodiment of the present invention;

FIG. 3 is a diagram illustrating an exemplary ACO recommender system according to an embodiment of the present invention; and

FIG. 4 is a diagram illustrating an exemplary apparatus for performing one or more of the methodologies presented herein according to an embodiment of the present invention.

### DETAILED DESCRIPTION OF PREFERRED EMBODIMENTS

As provided above, the identification of naturally-occurring accountable care organizations (ACOs) is an important step in transitioning a healthcare care system from fee-for-service to valued-based reimbursement. Provided herein is a systematic data-driven approach that discovers provider communities with promising features of high performing ACOs, e.g., high in-community utilization in both visit frequency and costs.

The definitions of some terms/abbreviations used throughout the present description are now provided. NPI refers to a national provider identifier (e.g., physicians, specialists, etc.). With the present techniques, NPIs are not used per se since any healthcare provider identification is sufficient. Each “contractual organization” (or simply “organization”), also referred to herein as a “site,” is composed of one or more NPIs. Each ACO is composed of one or more contractual organizations which share responsibility for outcomes of a set of patients (“shared savings” and “value-based payment” are synonymous terms which can be used to refer to the contracts that can be formed in accordance with the present techniques). To use a simple example, in the typical healthcare context a patient is treated by a physician. The physician, an NPI, can be part of at least one contractual organization (e.g., a physician can work in private practice, as well as for a hospital, etc. and thus may be part of more than one organization). The contractual organization can in turn be part of at least one ACO. Thus, the ACO is essentially a list of healthcare providers, each having an association with at least one organization/site. A goal of the present techniques is to produce a ranked list of organizations/sites to add to an ACO as an output of the present process. This list of organizations/sites can then be used by entities, such as insurance companies, to help them determine which organizations/sites (i.e., based on the ranking) the entities should write contracts with in the interest of value-based healthcare.

Namely, as will be described in detail below, the present process starts with insurance claims data, and observes their effects on the ACO with visualization. For instance, it can first be determined which healthcare providers belong to pre-existing informal networks of shared patients (e.g., a graph of NPIs can be constructed from health insurance claims data based on shared patients in order to apply community detection methods). Next, organization-level relationships can be found based on individual NPI relationships. Provider communities of a specified appropriate size with a specified amount of shared patient care can be created. The resulting ACOs improve coordination of care by promoting shared responsibility for the outcomes of a set of patients.

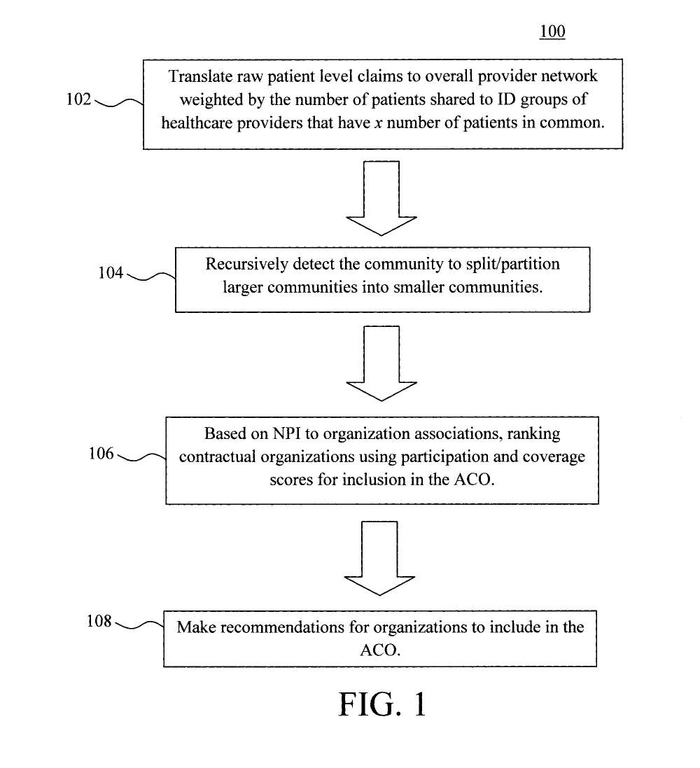

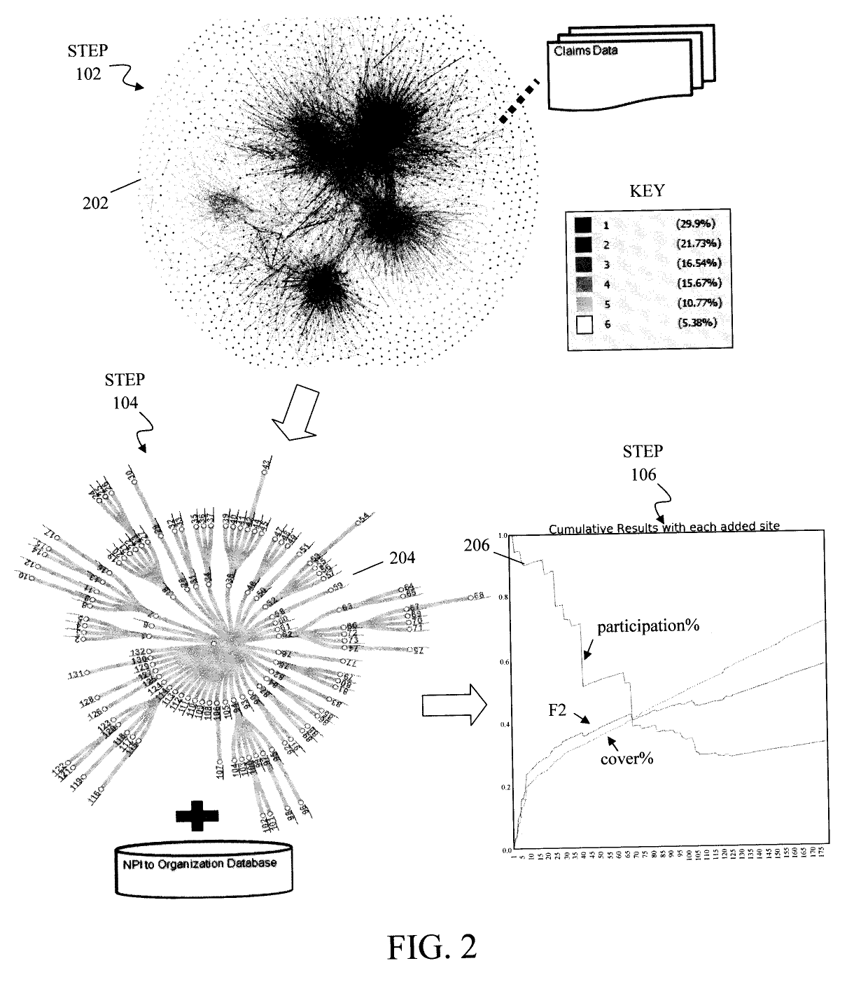

A detailed description of the present techniques is now provided by way of reference to FIGS. 1 and 2. FIG. 1 outlines the process (methodology 100 for forming ACOs) and FIG. 2 provides a schematic depiction of the techniques employed at each step of FIG. 1. Thus, FIGS. 1 and 2 will be described together.

Referring to FIG. 1, in step 102 the raw patient level insurance claims are translated to overall provider network weighted by the number of patients shared. The idea here is to determine from the raw insurance claim data (specifically the insurance claim data relating to patients and healthcare providers (e.g., physicians) treating the patient) collaborations between healthcare providers. Namely, step 102 identifies the number of patients the healthcare providers (e.g., the physicians) share in common. From this data, groups of healthcare providers can be identified that have x number of patients in common, wherein x=1, 2, 3, etc. For instance, one grouping might contain healthcare providers in the network who have one patient in common, another group might contain those healthcare providers having two patients in common, and so on.

One can visualize this collaboration relationship as shown in FIG. 2. For instance, referring briefly to the illustration of STEP 102 in FIG. 2, patient utilization history (i.e., data relating to which healthcare providers patients have seen in the past) as determined via the Claims data is used to create a chart 202 of the network of NPIs weighted by the number of patients shared. Specifically, each dot in chart 202 represents a healthcare provider (e.g., a physician, specialist, etc.) in the network. Each dot is shaded according to the number of patients the healthcare provider corresponding to that dot shares with other healthcare providers (as represented by other dots) in the network. For instance, using the KEY to the right of chart 202 it is shown that about 30% of the healthcare providers in the network share one patient in common (i.e., exactly one patient has been treated by those two healthcare providers), about 22% of healthcare providers share two patients in common, and so on. The corresponding shading used for the graph 202 is provided in the key. As can be seen from graph 202, the number of healthcare providers in each shade grouping is quite large. Thus, if one were simply to create ACOs from this initial processing of the raw data, then there would be only a few (in this example 6) different groups, some with hundreds of healthcare providers. Therefore, the next task is to subdivide each of these groups.

Namely, referring back to methodology 100 of FIG. 1, in step 104 a community detection algorithm is then used in a recursive manner to subdivide/partition the groups (from step 102) into smaller and smaller communities, eventually ending up with groups having high levels of collaboration. The goal is to detect smaller communities within the groups. With the recursive application, community size is easier to control. Community detection algorithms are described, for example, in A. Lancichinetti et al., “Community detection algorithms: a comparative analysis,” Physical Review E 80, 056117 (November 2009), the contents of which are incorporated by reference as if fully set forth herein. The community detection algorithm is applied recursively to the smaller groups each time. For example, in the first iteration two groups are produced, say Group A and Group B. In the next iteration, the community detection algorithm is then applied to Group A and Group B separately, and so on.

According to an exemplary embodiment, some additional constraints are imposed on the community detection process. For instance, one constraint might be that each community size has to be at least x number (i.e., each community has to contain at least x number of healthcare providers). In that manner, one can derive communities of a meaningful size rather than simply groups of nominally few individuals. Another relevant constraint might be a certain ratio of specialists to primary healthcare providers. A primary care provider is often the first contact for a patient, and the provider who will provide continued care for the patient. During their treatment, primary care providers might refer the patient to a specialist for various specialized care. The desired ratio of specialists to primary healthcare providers can vary depending on the situation and objectives of the patient. For instance, for many patients a low ratio of specialists to primary healthcare providers might be preferable since it means that the patient will have more primary physicians to pick from. On the other hand, for a patient with special requirements, a higher ratio of specialists to primary healthcare providers might be preferable to have a greater range of specialists. According to an exemplary embodiment, this ratio of specialists to primary healthcare providers is applied as a range specification (e.g., a ratio of specialists to primary healthcare providers of from X value to Y value).

Yet another relevant constraint might be in-community utilization. In-community utilization looks at, of all the services a patient has received, what percentage of the care happened in the patient's assigned community. A high in-community utilization means that the community has been designed well, meeting all the needs of the patient. Again, this constraint can be applied as a range specification (e.g., a percentage of in-community care of from X % to Y %).

One can visualize this recursive partitioning step using the community detection algorithm as shown in FIG. 2. For instance, referring briefly to the illustration of STEP 104 in FIG. 2, the partitioning process is shown as a tree graph 204 having a root and concentric leaf nodes. The value given at each leaf node represents a (unique) community identification (ID) number. As one spirals out from the center of the graph 204, the groups get smaller and smaller (i.e., the rings represent each iteration of the partitioning process as one moves from the center of graph 204, outward). Thus each time the partitioning process is iterated (as described above), the number of healthcare providers in each group gets successively smaller. Due to this recursive partitioning process, the leaf nodes in the tree graph 204 contain the most collaborative groups. Namely, the closer the node is towards the center of the graph 204, the more providers it contains. The idea here is that, in most cases, rather than having a single contract for a large number of providers, it is desirable to have tailored contracts for smaller number of providers; hence, the need for the recursive algorithm.

Up to this point in the process, the analysis has been at the provider level, meaning that (as described in detail above) it has focused on the healthcare providers themselves and the patients they share in common. However, a given healthcare provider can practice at multiple sites, also referred to herein as contractual organizations (see above). For instance, a physician can be in private practice, but can also treat patients at a hospital and/or at a clinic. Thus, a more comprehensive understanding of value-based healthcare can be achieved by next evaluating the groups (communities) that were created in step 104 (via the community detection process) at the contractual organization level. For instance, an NPI-to-contractual organization database (specifying which healthcare providers are associated with each contractual organization/site—for example a list of physicians who practice at a given hospital, clinic, etc.) can be used to link the healthcare providers in the groups/communities from step 104 to one or more contractual organizations. See, e.g., FIG. 2 (NPI to Organization Database).

The ultimate goal will be to determine, based on this NPI to organization association, which contractual organizations to include in the ACO (i.e., this will be done at the organization-level) so as to best represent the detected communities from step 104. As provided above, the ACO is essentially a list of healthcare providers. Further, since the process begins (as described above) with claims data—which has only healthcare provider-level information—then up through step 104 the process represents the resulting ACO as a list of healthcare providers in the detected communities. Another way to look at it is that the ACO of healthcare providers based just on the raw claims data represents an initial/raw ACO of the healthcare providers. After obtaining the raw ACO as a list of healthcare providers, the next task will be to distill it, as accurately as possible, the final ACO (at the organization/site level). Thus, since the goal is to write contracts at the site/organization-level, the ACO must be approximated at this stage (e.g., based on the list of healthcare providers in the communities detected in step 104—i.e., the raw ACO), and one way of measuring the performance of the approximation is by looking at its F-score.

For instance, if one were to evaluate contractual organizations for inclusion in the ACO based simply on the provider communities (determined in step 104) and those providers' associations with contractual organizations, then some contractual organization might be over-represented while others are under-represented. This is because, as provided above, healthcare providers can practice at multiple sites. To use a simple example to illustrate this concept, if one healthcare provider in the group (Physician A) practices at multiple sites (e.g., at multiple hospitals and/or clinics in addition to a private practice) and another healthcare provider in the group (Physician B) has only a private practice and does not go to any hospitals, then the sites/organizations contracted with Physician A might be over-represented, while those contracted with Physician B might be under-represented.

Thus, referring back to FIG. 1, based on the provider communities (detected in step 104) and those providers' associations with contractual organizations, in step 106 measures are computed to represent contractual organizations as they relate to the provider communities (detected in step 104). Suitable measures include, but are not limited to, participation percentage, cover percentage, and modified F2 score.

In general, an F-score is used to measure accuracy. Here a modified F-score is used to rank order the contractual organizations to include in an ACO. Basically, a score is computed for each of the contractual organizations being added to the ACO. The scores can then be used to rank the contractual organizations and the contractual organizations having the top x scores are included in the ACO. The modified F-score used herein is based on participation percentage (participation %) and cover percentage (cover %) scores. For instance,

F-score=(1+μ²)*((participation %*cover %)/(μ²*participation %+cover %)),

wherein,
participation %=(# of organization NPIs in common with ACO)/(# of NPIs in organization)
cover %=(# of organization NPIs in common with ACO)/(# of NPIs in ACO)

Thus, when evaluating a given one of the contractual organizations for inclusion in the ACO, the participation % looks at the number of healthcare providers the contractual organization has in common with the ACO as a function of the number of healthcare providers in the contractual organization, while the cover % looks at it as a function of the number of healthcare providers in the ACO. Since the ACO is essentially a list of providers, then the healthcare providers in the ACO can be based on the list of healthcare providers in the communities detected in step 104. Then what the participation % and cover % are measuring is how well the site-level “approximation” represents the original ACO provider list. The parameter μ is tunable. By way of example only, the parameter μ can be changed by the end user (e.g., end user entity such as the insurance company). The higher the value of parameter μ the more emphasis is placed on achieving high coverage. Namely, as shown in the equations above, the parameter μ can be used to tune participation %/cover % based, e.g., on an end-user's preferences.

One can visualize this organization-level analysis as shown in FIG. 2. For instance, referring briefly to the illustration of STEP 106 in FIG. 2, a graph 206 is shown of participation %, cover %, and F2 score for each site (i.e., contractual organization) added to the ACO. The values on the x-axis of graph 206 represent the number of organizations/sites that have been included in the ACO so far. The graph 206 is designed so that the end user (e.g., end user entity such as the insurance company) will have a particular threshold in mind and can just pick the first time that threshold is reached as the cut-off point for adding organizations/sites to the ACO. Another way the cut-off point can be selected is by looking at the intersection of the cover % and participation % lines.

Of course, not every contractual organization should be included in the ACO. Advantageously, the present techniques provide a data-driven approach to meaningfully evaluate organizations for inclusion in the ACO. For instance, based on the modified F-score computed for each contractual organization, the contractual organizations can be ranked. Then the organizations with the top y scores can be included in the ACO. Namely, the end-user can look at the F-score line in graph 206 and pick the first time a threshold is achieved. This process enables use of a single number (score) to understand the effect of including a contractual organization in the ACO.

Referring back to FIG. 1, based on the ranking obtained in step 106, recommendations are made in step 108 as to which contractual organizations to include in the ACO. For instance, as provided above, the recommendation can be simply to include the top y ranked contractual organizations in the ACO. The value of y can be predetermined by the user.

By way of example only, the end user might be an insurance company that employs the recommendations to build ACO networks of organizations. The insurance company can leverage the present techniques to better understand the effect of including various contractual organizations in the network. Namely, as provided above, the participation percentage, cover percentage, and modified F-score can be computed for each candidate organization's inclusion in the ACO. For instance, adding or removing a given organization/site from the ACO will change the overall F-score.

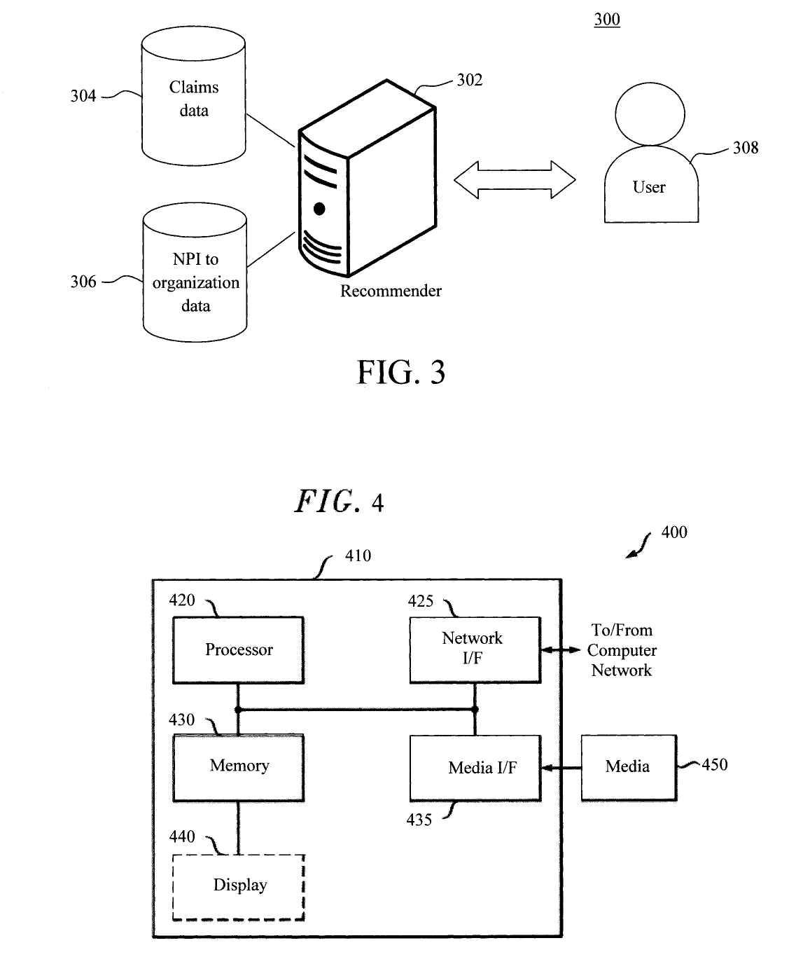

Also provided herein is a recommender system 300 for building ACOs. See FIG. 3. As shown in FIG. 3, system 300 includes a recommender engine 302. Recommender engine 302 (e.g., a server) is configured to perform the above-described steps of methodology 100. As provided above, this includes using raw claims data (obtained by recommender engine 302 from claims database 304) to identify groups of healthcare providers that have x number of patients in common instance, use community detection techniques to recursively partition the groups into smaller groups/communities, and then expanding the analysis out from the provider level to the organization level to find organizations for inclusion in the ACO that best represent the group/communities (using provider to organization data obtained from NPI to organization database 306). The recommender engine 302 can then make recommendations to a user 308 as to what contractual organizations to include in the ACO (i.e., so as to best represent the detected groups/communities) based, for example, on a modified F-score ranking. The user can also set parameters for the recommendation engine 302, such as the constraints to be imposed on the recursive partitioning process (e.g., minimum community size, specialist/primary care physician ratio, etc.), the number of the top ranked organizations to include in the ACO, etc.

The recommender engine 302 can also provide the user with visualizations corresponding to the steps of the recommendation process, as is depicted, e.g., in FIG. 2. For instance, the recommender engine 302 can provide the user with graphs/charts depicting the data (at the provider and organization levels) used to arrive at the recommendations.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Turning now to FIG. 4, a block diagram is shown of an apparatus 400 for implementing one or more of the methodologies presented herein. By way of example only, apparatus 400 can be configured to implement one or more of the steps of methodology 100 of FIG. 1. For instance, apparatus 400 can be configured to serve as recommendation engine 302 in system 300.

Apparatus 400 includes a computer system 410 and removable media 450. Computer system 410 includes a processor device 420, a network interface 425, a memory 430, a media interface 435 and an optional display 440. Network interface 425 allows computer system 410 to connect to a network, while media interface 435 allows computer system 410 to interact with media, such as a hard drive or removable media 450.

Processor device 420 can be configured to implement the methods, steps, and functions disclosed herein. The memory 430 could be distributed or local and the processor device 420 could be distributed or singular. The memory 430 could be implemented as an electrical, magnetic or optical memory, or any combination of these or other types of storage devices. Moreover, the term “memory” should be construed broadly enough to encompass any information able to be read from, or written to, an address in the addressable space accessed by processor device 420. With this definition, information on a network, accessible through network interface 425, is still within memory 430 because the processor device 420 can retrieve the information from the network. It should be noted that each distributed processor that makes up processor device 420 generally contains its own addressable memory space. It should also be noted that some or all of computer system 410 can be incorporated into an application-specific or general-use integrated circuit.

Optional display 440 is any type of display suitable for interacting with a human user of apparatus 400. Generally, display 440 is a computer monitor or other similar display.

Although illustrative embodiments of the present invention have been described herein, it is to be understood that the invention is not limited to those precise embodiments, and that various other changes and modifications may be made by one skilled in the art without departing from the scope of the invention.

What is claimed is:

1. A method for forming an Accountable Care Organization (ACO), comprising:
determining groups of healthcare providers that have x number of patients in common;
detecting communities in the groups using a recursive community detection process;
ranking contractual organizations of the healthcare providers based on how well the contractual organizations represent the communities; and
making recommendations for the contractual organizations to include in the ACO based on the ranking.

2. The method of claim 1, wherein the determining is performed using raw insurance claim data.

3. The method of claim 1, further comprising:
imposing constraints on the communities.

4. The method of claim 3, wherein the constraints comprise a set minimum number of the healthcare providers that needs to be included in each of the communities.

5. The method of claim 3, wherein the healthcare providers include both specialists and primary care providers, and wherein the constraints comprise a set ratio of the specialists to the primary care providers that needs to be included in each of the communities.

6. The method of claim 1, further comprising:
determining associations between the healthcare providers and the contractual organizations.

7. The method of claim 6, wherein at least one of the healthcare providers is associated with more than one of the contractual organizations.

8. The method of claim 1, further comprising:
assigning scores to each of the contractual organizations.

9. The method of claim 8, wherein the contractual organizations are ranked based on the scores.

10. The method of claim 8, wherein the recommendations for the contractual organizations to include in the ACO comprise the contractual organizations having y highest scores.

11. A non-transitory computer-readable program product for forming an ACO, the computer program product comprising a computer readable storage medium having program instructions embodied therewith which, when executed, cause a computer to:
determine groups of healthcare providers that have x number of patients in common;
detect communities in the groups using a recursive community detection process;
rank contractual organizations of the healthcare providers based on how well the contractual organizations represent the communities; and
make recommendations for the contractual organizations to include in the ACO based on the ranking.

12. The non-transitory computer-readable program product of claim 11, wherein the program instructions which, when executed, further cause the computer to:
impose constraints on the communities.

13. The non-transitory computer-readable program product of claim 12, wherein the constraints comprise a set minimum number of the healthcare providers that needs to be included in each of the communities.

14. The non-transitory computer-readable program product of claim 12, wherein the healthcare providers include both specialists and primary care providers, and wherein the constraints comprise a set ratio of the specialists to the primary care providers that needs to be included in each of the communities.

15. The non-transitory computer-readable program product of claim 11, wherein the program instructions which, when executed, further cause the computer to:
determine associations between the healthcare providers and the contractual organizations.

16. The non-transitory computer-readable program product of claim 15, wherein at least one of the healthcare providers is associated with more than one of the contractual organizations.

17. The non-transitory computer-readable program product of claim 11, wherein the program instructions which, when executed, further cause the computer to:
assign scores to each of the contractual organizations.

18. The non-transitory computer-readable program product of claim 17, wherein the contractual organizations are ranked based on the scores.

19. The non-transitory computer-readable program product of claim 17, wherein the recommendations for the contractual organizations to include in the ACO comprises the contractual organizations having y highest scores.

20. A system for forming an ACO, comprising:
a recommender engine configured to:
determine groups of healthcare providers that have x number of patients in common;
detect communities in the groups using a recursive community detection process;
rank contractual organizations of the healthcare providers based on how well the contractual organizations represent the communities; and
make recommendations for the contractual organizations to include in the ACO based on the ranking.
